(19) United States
(12) Patent Application Publication
(10) Pub. No.: US 2021/0330100 A1
(43) Pub. Date: Oct. 28, 2021

(54) Weighted Blanket with Far Infrared (FIR) Emitters and EMF Shielding

(71) Applicant: Tina Ureten, Milton (CA)

(72) Inventor: Tina Ureten, Milton (CA)

(21) Appl. No.: 17/233,397

(22) Filed: Apr. 16, 2021

(30) Foreign Application Priority Data
Apr. 24, 2020 (CA) 3079585

(51) Int. Cl. A47G 9/00; A47G 9/02; A61M 21/02; A61N 1/16; A61N 5/06

(52) U.S. Cl. CPC A47G 9/007; A47G 9/0223; A61M 2021/0022; A61N 1/16; A61N 5/06; A61M 21/02

(57) ABSTRACT

Provided are a weighted blanket and method of manufacturing. The weighted blanket has a laminated structure comprising: a bottom sheet; a top sheet having electromagnetic field (EMF) shielding embedded therein; a batting layer between the sheets; and Far-Infrared-emitting bioceramic beads. The blanket is quilted to retain the beads in quilted pockets. The blanket provides FIR emissions to the user, while protecting them from external EMF.

### RELATED APPLICATIONS

This application claims priority to Canadian Patent Application CA3079585 with filing date 24 Apr. 2020.

### FIELD OF THE INVENTION

The invention relates to blankets, particularly to weighted blankets covering a user and providing health and wellness benefits.

### BACKGROUND OF THE INVENTION

Recent technological advancements make tremendous changes in our lifestyles and health. Electronic devices have become part of daily life but the long-term health effects from non-stop exposure to those devices and electromagnetic signals have some negative effects on health and wellbeing. One health problem is sleep deprivation due to constant exposure to blue light. Sleep is one of the most critical factors for physical and mental health of the individuals and productivity of the whole society. Younger age onset of insomnia leads to many chronic health problems. Studies show that lack or deprivation of sleep can have profoundly negative consequences for the health, productivity and emotional/mental wellbeing of people.

Continuous exposure to electromagnetic fields (EMF) due to wireless technologies is associated with a rising rate of chronic illnesses. There is strong evidence that long-term exposure to certain EMFs is a risk factor for diseases such as certain cancers, cognitive dysfunctions and male infertility. Electromagnetic Hypersensitivity Syndrome (EHS) is a new disease due to EMF exposure recognized by health authorities.

Weighted blankets currently on the market are shown to improve sleep in people, particularly by reducing anxiety and creating a sense of snugness.

### SUMMARY OF THE INVENTION

The inventor has appreciated that many of the above problems can be alleviated by improving current blankets with layers of materials having properties intended to provide certain benefits.

According to one aspect of the invention there is provided a weighted blanket having a laminated structure comprising: a bottom sheet; a top sheet having electromagnetic field (EMF) shielding embedded therein; a batting layer between the sheets; and Far-Infrared-emitting bioceramic beads. The laminate structure is quilted together to join the layers and retain the beads in quilted pockets.

According to a second aspect of the invention there is provided a method of manufacturing a weighted blanket comprising: providing a first fabric layer; overlaying the first fabric layer with a batting layer; overlaying the batting layer with second fabric layer; distributing Far-Infrared-emitting bioceramic beads substantially evenly between the batting and one of the fabric layers; and quilting the layers to retain the beads in quilted pockets. One of the fabric layers comprises an electromagnetic field (EMF) shielding embedded therein.

Further aspects are set out in the attached claims.

### DESCRIPTION OF THE DRAWINGS

Details of preferred embodiments of the present invention are further shown in the attached drawings in which:

FIG. 1 is a perspective, exploded view of layers of a weighted blanket in accordance with embodiments the invention;

FIG. 2 is a side view through the cross-section A-A of FIG. 1;

FIG. 3 is a top view of the weighted blanket; and

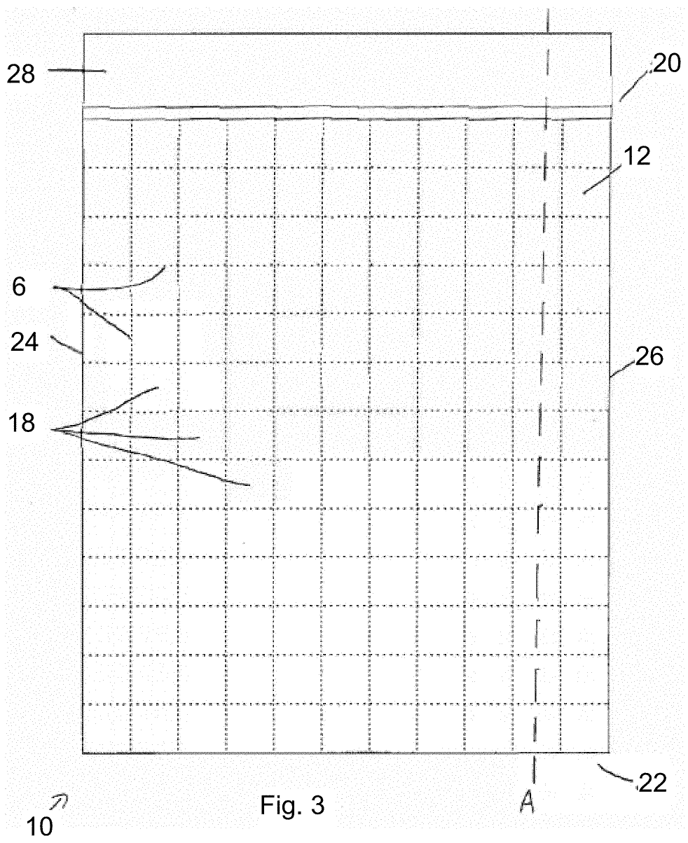

FIG. 4 is a flowchart for manufacturing the weighted blanket.

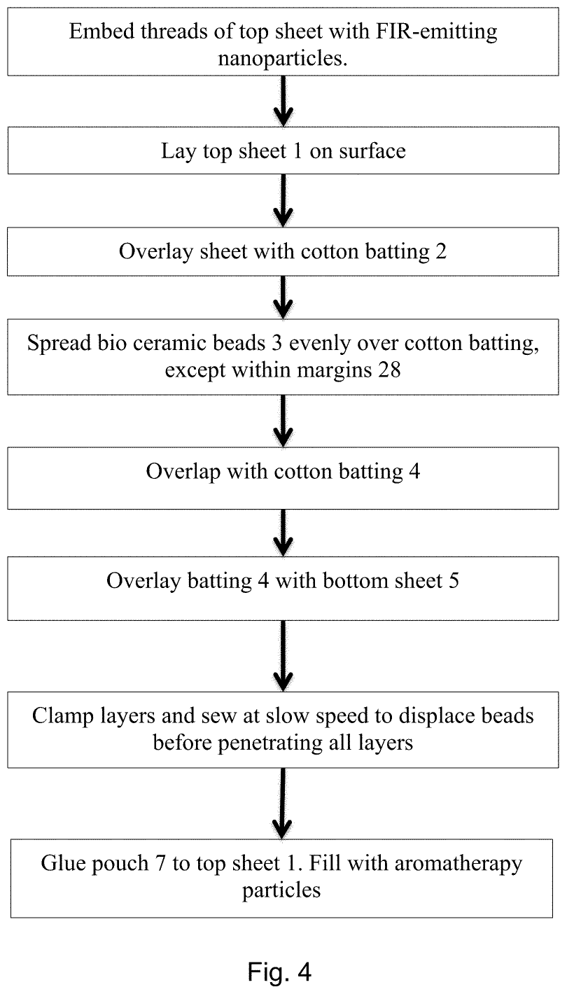

### DETAILED DESCRIPTION OF THE INVENTION

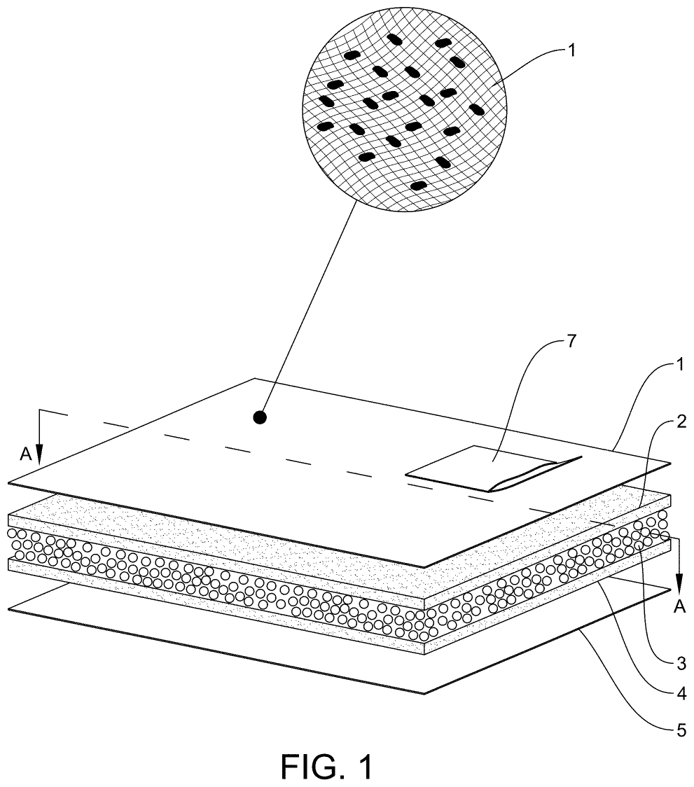

Referring to FIG. 1, a blanket made in accordance with the present invention, consists of a roughly rectangular blanket having a laminate structure, including an upper sheet 1, bioceramic beads 3, and a lower sheet 5, which are quilted together by a pattern of bindings 6 to form a plurality of enclosed pockets 18 formed between the upper and lower layers of fabric sheets to hold the beads. One or more middle battling layers 2, 4, may be used to hold the beads in place.

The layers have equal widths and lengths. The first layer 5 of the fabric is the closest to the user's skin. This fabric may be produced using nano technology to embed far infrared emitting minerals of nanometer size into the fibers. The close contact of this layer of fabric with the user provides health effects associated with far infrared rays during sleep. The second fabric sheet 1 forms the most top level of the blanket furthest from the body. This fabric is produced with EMF blocking material embedded within the threads of the fabric to diminish the exposure to EMF during sleep. For example, fabrics may have particles of silver, copper, or steel embedded within the threads that absorb EMF radiation, depending on the conductivity, permeability, thickness, and aperture of the material used.

Above the first fabric sheet 5 is a fluffed, aerated batting layer 4, such as non-glue cotton, wool, bamboo or polyester layer. This layer may be produced by using natural fibers, which create air pockets and ducts therethrough to allow moisture wicking, air circulation and some heat insulation. After carding the raw fibers, they are run through air ducts to make them fluffy and soft. Then they are pressed with heat to bring them to a specific thickness. This layer also prevents the movement of the next layer—bioceramic beads 3.

The beads may be generally spherical. These beads may be entrained within a batting layer or sandwiched between two batting layers. The bioceramic beads comprise one or more minerals that emit FIR when irradiated with some form of energy, such as body heat. The mineral content may include stones such as tourmaline, Maifan or Muyu Stones, wherein the content of the mineral portion in the bead by mass is 25-35%, 25-35%, and 5-10% respectively. Alternatively the mineral content may include: sepiolite, quartz, kaolin or other clays, each 3-10% by mass of the bead.

The bioceramic beads may be a mixture of tourmaline ceramic beads, maifan stone ceramic beads, titanium dioxide ceramic beads, graphene ceramic beads and silver ion ceramic beads. Tourmaline is a naturally occurring gemstone comprising boron silicate minerals. It generally includes elements such as aluminium, iron, magnesium, sodium, lithium, or potassium. Maifan Stone is a mineral rock which generally includes the elements calcium, iron, zinc, magnesium, copper, and selenium. Maifan Stone has strong adsorption ability for heavy metal ions and reduces bacteria concentration, by inhibiting its reproduction speed.

Sewing all the layers in a quilted and continuous pattern forms the blanket and it keeps the bioceramic beads in the internal space in the form of small closed pockets 18. Each of these quilted pockets carry an equal amount of FIR emitting bioceramic beads. The total weight of the blanket is mostly provided by the bioceramic beads and ranges from 8 kg to 15 kg for an adult, increasing with the user's body size. The exposure of FIR and blocked EMFs provides a healthy condition during sleep and for better health and wellness.

The pattern of bindings preferably consists of stitches, however, other methods of binding may be used such as gluing, bonding or fabric welding. A regular-shaped, contiguous pattern of binding, such as a diamond pattern or grid pattern, is preferably used, so that the pockets are formed with adjacent pockets in contact with each other and the beads are evenly spread. It is possible to employ other pattern bindings, such as a hexagonal pattern, fish scale pattern which are capable of producing a contiguous pattern of pockets of approximately equal dimensions.

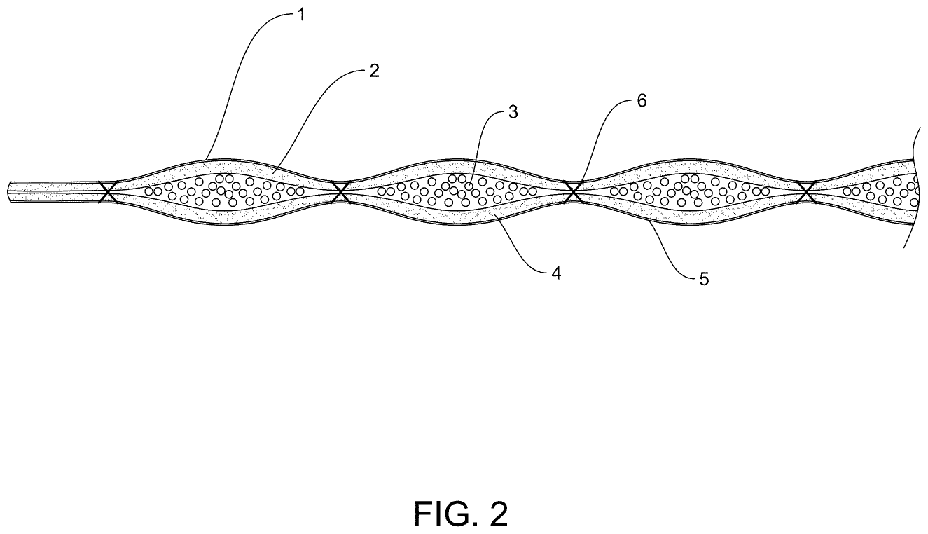

Referring now to the cross-section FIG. 2 through line A-A, the pockets separated by bindings (stitches) 6 are relatively small compared to the blanket, preferably in the range of 5 cm to 15 cm per dimension. Preferably the total cumulative total weight of all of the beads in the blanket depends on the body weight of the person intending to use the blanket. For the children size blankets the weight can be between 4 to 10 KGs, the adult size blankets can be between 8 to 15 KGs.

The weight in the blanket brings pressure to the deep pressure touch points and acts like a massager to release serotonin hormone. Serotonin is known for its relaxation effect on the body and its end product is melatonin, the sleep hormone. Serotonin is the body's “feel good” substance. It contributes to well being and happiness and can regulate mood, mental health, memory and sleep.

One of the bottom or upper sheets may have a closable envelope or pocket 7 for holding material therein, preferably aromatherapy material. Elongated envelope 7 has an opening which is covered over by a flap, which can close over the opening and held in place by closure means. Closure means preferably consists of a strip of hook and loop fastener, such as Velcro™, but may comprise an elongated zipper or a row of buttons, snaps or some other closure. The fabric forming envelope is sufficiently porous that scent emanating from a satchel can pass therethrough. Contained within the pocket may be a satchel which is impregnated with aromatherapy sheets, seeds or aromatherapy oils. Minuscule amounts of different aromatherapy oils can be used. By placing satchel in envelope 7 positioned near one end of the blanket, the blanket can be worn over the user with the envelope close to the user's head, thereby releasing aromatherapy vapors/scent close to the user's nose provides the relaxing and calming effect and enhances the sleep quality.

The therapeutic benefit of using aromatherapy (botanical) extracts has been recognized by mankind for ages. In more modern history, companies have produced commercial lines of extracted essential oils for topical and aroma therapeutic use. Aromatherapy Oils are the highly concentrated, volatile, aromatic essences of plants. They are extracted directly from a variety of flowers, fruits, leaves, barks, roots, herbs, and spices. Most common essential oils known for their therapeutic sleep benefit are lavender, chamomile, sage, ylang ylang, bergamot, marjoram and cedarwood.

For example, lavender, roman chamomile, and neroli mixed in a 6:2:0.5 ratio is effective at reducing stress and improving the sleep of patients. Alternatively mixed lavender, ylang-ylang, marjoram, and neroli may lower blood pressure and improve sleep of users with hypertension. Mixtures of lavender, basil, juniper, and marjoram may increase sleep quality.

The present blanket may significantly enhance the benefits of weighted blankets by adding materials designed to provide far infrared ray (FIR) to the user during sleep. Infrared radiation can be categorized into three groups according to wavelength, namely: near infrared (NIR, 0.8-1.5 μm); middle infrared (MIR, 1.5-5.6 μm); far infrared (FIR, 5.6-1000 μm).

The far infrared ray is the most beneficial of all infrared rays. These energy waves can penetrate the body up to 10 cm and elevate the surface temperature just slightly enough to increase the body's blood flow via expanding the capillaries. They also boost blood oxygenation and clean the toxins. Therefore, the healing power of these rays can benefit muscles, cells, blood vessels, lymph glands, organs and boost cleaning the inflammation and metabolic waste. Not only is FIR absorbed by the human body but it is also emitted by the body in the form of body radiation.

The present blanket includes FIR-emitting minerals in at least one of its layers: bottom layer 5, batting layers 2, 4 and bioceramic layer 3. Particles of tourmaline, germanium, graphene, quarts, titanium, jade, maifan stone, graphene, bamboo charcoal and similar minerals are sources that emit FIR when irradiated. These materials, preferably provided as nanoparticles, may be incorporated into the fabric material such as cotton, wool, polyester, rayon or blended fibers. The end product has effective therapeutic benefits for the users when applied to the skin.

The outer layer fabric 1 overlays the non-glue compressed cotton layer to support the weight of the bioceramic beads in the inner layer. The non-glue cotton layer may also comprise FIR-emitting minerals to boost the effect of FIR on the body. The bioceramic beads are sandwiched between batting layers 2, 4 to stabilize the bioceramic beads. In addition to FIR's good effect on blood circulation, vitality, hormonal balance and immune system, histological findings revealed greater collagen regeneration and infiltration of fibroblasts that expressed transforming growth factor-β1 (TGF-β1). TGF-β1 accelerates wound healing with FIR irradiation.

The bioceramic beads 3 preferably comprise mixing different kinds of FIR emitting beads. The sphere shaped bioceramic beads are preferably between 1 to 5 mm in size and their weight are heavy enough to alter tactile inputs of the body and increase serotonin level in the brain which has strong relaxation and anti-depressive effect. The natural clay forming the ceramic beads has a porous surface which can easily absorb the moisture to maintain dryness and air circulation between the layers of the blanket. Therefore the present blankets may provide thermal regulation during sleep.

Preferably a minimum of 10% of the beads are silver ion bioceramic beads due to the special properties of silver. Silver's strong antibacterial/antimicrobial effect is well documented in the medical literature and silver ions keep the blanket free of odour producing bacteria. Silver ions also provide anti-static environment and may accelerate the healing of skin from cuts and wounds. Another health benefit from silver ions is blockage of environmental electro-magnetic radiation.

The top layer 1 of the present blanket is made with a shielding fabric which can block electro-magnetic radiation coming from environmental sources. Electromagnetic Fields (EMFs) are radiation and include everything from cosmic rays through visible light to the electric and magnetic fields associated with electricity.

Currently, the exposure to various types of EMFs is significantly increased due to continually usage of cell phones and wireless devices in addition to other sources such as power lines, computer devices, televisions, radios and telecommunication devices. EMFs have high penetration power, and they are capable of moving charged particles, such as the electrons and ions of large macromolecules and polymers.

Developments in nano-technology enables embedding the EMF blocking particles into the thread of the fabric (see inset of FIG. 1). The EMF shielding particles, such as silver, nickel, copper, iron or steel particles can be combined with the fiber without changing the flexibility of the fabric. The present blanket uses the shielding fabric for the outermost layer of the weighted blanket to block the EMFs for the users.

In an alternative embodiment, a substantially continuous metal foil is comprised within the top layer. The foil may be laminated onto the top sheet. This provides continuous EMF shielding but reduces the breathability of the overall blanket. Therefore thru-holes may be made in the foil to improve breathability while providing shielding.

Electro hypersensitivity syndrome (EHS) is a new medical diagnosis which results from continued involuntary exposure to EMF radiation and increasingly causes concern by health care professionals. The WHO and medical authorities list the symptoms of EHS as fatigue, concentration difficulties, sleep disturbance, dizziness, nausea, heart palpitation, headache skin prickling, burning sensations and rashes, pain and ache in muscles. The prevalence of EHS has ranged from 1.5% to 13.3%. (Ref: https://www.ncbi.nlm.nih.gov/pubmed/26372109).

As such, electromagnetic hypersensitivity is becoming a more serious concern especially at school age children, it is getting increasingly difficult for those who suffer from the condition to find any respite—it is almost impossible in modern society to find locations that do not have detectable levels of EMF radiation. However decreasing the exposure can be sufficient to eliminate mild and moderate symptoms.

The present blanket has the most top layer made with shielding fabric in order to decrease the exposure time and density to EMFs during the sleep hours. Therefore the blankets can diminish hazardous risk from exposure to EMFs and decreases the symptoms of EHS symptoms. Shielding fabric can be made by using EMF blocking particles incorporated into cotton, wool, polyester or blended fabric materials. The textile's softness, durability, elasticity, water resistance and breathability can be enhanced with the known production methods. If the blanket is kept between the electric and electronic device and the body, the electromagnetic waves from the device will not be able to reach the body. The fabric also keeps a balanced charge in fabric to reduce static buildup and its properties don't change with washing.

The present blanket 10 thus provides two different functional layers for EMF shielding and FIR emission, while additionally providing weight and aromatherapy. The overall effect promotes the easy onset of sleep, longer and deeper sleep experience and enhances wellness for the user.

In preferred embodiments, there are one or more margins 28 at the periphery of the blanket that have no beads. These margins make it easier to grasp the blanket. As these marginal areas tend not to lie over the user during sleep, they do not need to provide benefits of weight or FIR emissions. The margins may be 5-15 cms wide and may extend the whole of an edge, and may be present on all edges 22, 24, 26 of the blanket. During manufacture, the marginal areas receive substantially fewer or preferably no beads and do not need to be quilted.

A specific embodiment of the present invention has been disclosed; however, several variations of the disclosed embodiment could be envisioned as within the scope of this invention. It is to be understood that the present invention is not limited to the embodiments described above, but encompasses any and all embodiments within the scope of the following claims. It will be appreciated that the present invention is developed to improve users' sleep and their health and wellness but is not limited to embodiments with the health benefits discussed herein. The benefits of certain aspects of preferred embodiments have been disclosed in the following references:

Vatansever F., Michael R. Hamblin. Far infrared radiation (FIR): its biological effects and medical applications. Photonics Lasers Med. 2012 Nov. 1; 4: 255-266.

Chang Y, Liu Y P, Liu C F. The effect on serotonin and MDA levels in depressed patients with insomnia when far-infrared rays are applied to acupoints. Am J Chin Med. 2009; 37(5):837-42.

1. A weighted blanket having a laminated structure comprising:
a bottom sheet;
a top sheet having electromagnetic field (EMF) shielding embedded therein;
a batting layer and Far-Infrared-emitting bioceramic beads between the sheets, wherein
the laminate structure is quilted together to join the layers and retain the beads in quilted pockets.

2. The blanket of claim 1, wherein the bioceramic beads have a cumulative weight of 8 to 15 kgs.

3. The blanket of claim 1, further comprising a second batting layer between the sheets, the two batting layers arranged to sandwich the ceramic beads therebetween.

4. The blanket of claim 1, wherein the batting layer(s) is made of a non-glue cotton having air pockets that entrain the beads.

5. The blanket of claim 1, wherein the top sheet comprises metal threads or metal nanoparticles embedded on fabric threads to block EMF waves, the metal preferably selected from one of: silver, iron, steel, nickel or copper.

6. The blanket of claim 1, wherein the bottom sheet comprises FIR-emitting materials.

7. The blanket of claim 1, wherein the FIR-emitting bioceramic beads comprise one or more of: tourmaline ceramic beads, alkaline ceramic beads, titanium dioxide ceramic beads, graphene ceramic beads, maifan stone ceramic beads, negative ion ceramic beads, silver ion ceramic beads, sepiolite, quartz, kaulin, and charcoal.

8. The blanket of claim 1, further comprising a closable aromatherapy pocket, preferably located on the top sheet, preferably wherein the aromatherapy pocket is made of a porous material to permit passage therethrough of aerosolized aromatherapy particles.

9. The blanket of claim 1, wherein one or more margins of the blanket do not include beads, preferably wherein the margin has a width between 5 cm and 15 cm.

10. A method of manufacturing a weighted blanket comprising:
providing a first fabric layer;
overlaying the first fabric layer with a batting layer;
overlaying the batting layer with second fabric layer;
distributing Far-Infrared-emitting bioceramic beads substantially evenly between the batting and one of the fabric layers; and
quilting the layers to retain the beads in quilted pockets,
wherein one of the fabric layers comprises electromagnetic field (EMF) shielding embedded therein.

11. The method of claim 10, further comprising embedding threads of the top sheet with metal nanoparticles, the metal preferably selected from one of: silver, iron, steel, nickel or copper.

12. The method of claim 10, further comprising providing a second batting layer over the first batting layer to sandwich the bioceramic beads therebetween;

13. The method of claim 10, wherein quilting is performed by sewing or gluing.

14. The method of claim 10, wherein the bioceramic beads have a cumulative weight of 8 to 15 kgs.

15. The method of claim 10, wherein the batting layer(s) is made of a non-glue cotton having air pockets that entrain the beads.

16. The method of claim 10, wherein the bottom layer comprises FIR-emitting materials.

17. The method of claim 10, wherein the FIR-emitting bioceramic beads comprise one or more of: tourmaline ceramic beads, alkaline ceramic beads, titanium dioxide ceramic beads, graphene ceramic beads, maifan stone ceramic beads, negative ion ceramic beads and silver ion ceramic beads.

18. The method of claim 10, wherein the FIR-emitting bioceramic beads comprises material selected from: sepiolite, quartz, kaulin, and charcoal.

19. The method of claim 10, further comprising providing a closable aromatherapy pocket on one of the fabric layers, preferably made of a porous material to permit passage therethrough of aerosolized aromatherapy particles.

20. The method of claim 10, wherein the beads are not distributed over one or more margins of the blanket, preferably wherein the margin has a width between 5 cm and 15 cm.
